SEQUENCE LISTING (27 sequences)

SEQ ID NO: 1 - protein, 356 aa, Zea mays
MACLTDLVNLNLSDNTEKIIAEYIWIGGSGMDLRSKARTLSGPVTDPSKLPKWNYDGSSTGQAPGEDSEVILYPQAIFKDPFRRGNNILVMCDCYTPAGEPIPTNKRYNAAKIFSSPEVAAEEPWYGIEQEYTLLQKDTNWPLGWPIGGFPGPQGPYYCGIGAEKSFGRDIVDAHYKACLYAGINISGINGEVMPGQWEFQVGPSVGISSGDQVWVARYILERITEIAGVVVTFDPKPIPGDWNGAGAHTNYSTESMRKEGGYEVIKAAIEKLKLRHREHIAAYGEGNERRLTGRHETADINTFSWGVANRGASVRVGRETEQNGKGYFEDRRPASNMDPYVVTSMIAETTIIWKP

SEQ ID NO: 2 - DNA, 5299 bp, Artificial Sequence
  proCsVMV-OsActin_intron-ZmGS1b-terminator ZmGS1b-terminator NOS-proZmRbcS-ZmGS1b-terminator ZmGS1b- terminator NOS
aaggtaattatccaagatgtagcatcaagaatccaatgtttacgggaaaaactatggaagtattatgtgagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtccctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagacgataagcttgatatcgaattcctgcaggtcgactctagaggatccacccctgtcgcaccgcagccgccggccatggcctgcctcaccgacctcgtcaacctcaacctctcggacaacaccgagaagatcatcgcggaatacatatggatcggtggatctggcatggatctcaggagcaaagcaaggaccctctccggcccggtgaccgatcccagcaagctgcccaagtggaactacgacggctccagcacgggccaggcccccggcgaggacagcgaggtcatcctgtacccgcaggccatcttcaaggacccattcaggaggggcaacaacatccttgtgatgtgcgattgctacaccccagccggcgagccaatccccaccaacaagaggtacaacgccgccaagatcttcagcagccctgaggtcgccgccgaggagccgtggtatggtattgagcaggagtacaccctcctccagaaggacaccaactggccccttgggtggcccatcggtggcttccccggccctcagggtccttactactgtggaatcggcgccgaaaagtcgttcggccgcgacatcgtggacgcccactacaaggcctgcttgtatgcgggcatcaacatcagtggcatcaacggggaggtgatgccagggcagtgggagttccaagtcgggccttccgtgggtatatcttcaggcgaccaggtctgggtcgctcgctacattcttgagaggatcacggagatcgccggtgtggtggtgacgttcgacccgaagccgatcccgggcgactggaacggcgccggcgcgcacaccaactacagcacggagtcgatgaggaaggagggcgggtacgaggtgatcaaggcggccatcgagaagctgaagctgcggcacagggagcacatcgcggcatacggcgagggcaacgagcgccggctcaccggcaggcacgagaccgccgacatcaacacgttcagctggggcgtggccaaccgcggcgcgtcggtgcgcgtgggccgggagacggagcagaacggcaagggctacttcgaggaccgccgcccggcgtccaacatggacccctacgtggtcacctccatgatcgccgagaccaccatcatctggaagccctgagggctaaggcggccgttccgtcgcgttgcgttgcgttgcagggtccccgaagcgattgcaaagccattgttccttctgttccgtttgcttattattatcattattatcatctagctagatcatccgggggtcaggtcgtcgtggtgtgccaaaacagaacacagaaagaagaagaaaaaaaaacaagacgtgtggcgttaatgttatgtcatccaataatggccgtaccacacttgtggtacctttcagtaacttcgttgcccggatcctctagagtcgacctgcagcccgggggatccaattcccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagtccctttaatctggcgctagatctgcatccgcggcttgcaaagataaatggcacatttagtgtgttattttgcaatacctttcatagtagatatccttaaatgcagttttaggcatgtttgggtaattaaataacatttttaggaggagttttagatttacctttctttcgtgatgactgatgacagacgtggggaattcaaatgcaactctagcgaaagttcatatatttttcataaatagctgaggctggggtaattattttttttgtagaaaaatagaataggtggaatggttggggaaggcgtaggcgctcgtggacgacgcccgataaaagacaagaggcggaattgccatgaattcgaggtagctaagtaaggcgcatatatatgccaaaaaattctactgtcactttccaatttcaatgcgctgccaaacaagccatcctggaaactgacttgaattcagcccaattctgtagatccaaacagggccggcgtcagtgcctcaggtgagagagcagcagacgatgcaaagagccaaaactgcaagcagacgcagccgaagccgaagccgaagcccaagcccaaaactgttttgtctttgcccagaaccgcgacgagcctaaactgcgcttcctcctatctacaagtccctggcacatcacgcatagtccaacctaggcgcgcaggcgataaggcgcgccacggggacgcgacatgtggtggcggacgcgatcaggatagggccaggctggccgggcgcggccacgggagaacggtggccactcgtcccacatccgcttcgtcctgtcctgtactgcgtcctgcccccaacgagagccggagccggccatcccgtcgcacactctccccctctatatatgccgtcggtgtgggggagcctactacaggacgacccaagcaagcaagcaagcagcgagtacatacatactaggcagccaggcagccaccatggcctgcctcaccgacctcgtcaacctcaacctctcggacaacaccgagaagatcatcgcggaatacatatggatcggtggatctggcatggatctcaggagcaaagcaaggaccctctccggcccggtgaccgatcccagcaagctgcccaagtggaactacgacggctccagcacgggccaggcccccggcgaggacagcgaggtcatcctgtacccgcaggccatcttcaaggacccattcaggaggggcaacaacatccttgtgatgtgcgattgctacaccccagccggcgagccaatccccaccaacaagaggtacaacgccgccaagatcttcagcagccctgaggtcgccgccgaggagccgtggtatggtattgagcaggagtacaccctcctccagaaggacaccaactggccccttgggtggcccatcggtggcttccccggccctcagggtccttactactgtggaatcggcgccgaaaagtcgttcggccgcgacatcgtggacgcccactacaaggcctgcttgtatgcgggcatcaacatcagtggcatcaacggggaggtgatgccagggcagtgggagttccaagtcgggccttccgtgggtatatcttcaggcgaccaggtctgggtcgctcgctacattcttgagaggatcacggagatcgccggtgtggtggtgacgttcgacccgaagccgatcccgggcgactggaacggcgccggcgcgcacaccaactacagcacggagtcgatgaggaaggagggcgggtacgaggtgatcaaggcggccatcgagaagctgaagctgcggcacagggagcacatcgcggcatacggcgagggcaacgagcgccggctcaccggcaggcacgagaccgccgacatcaacacgttcagctggggcgtggccaaccgcggcgcgtcggtgcgcgtgggccgggagacggagcagaacggcaagggctacttcgaggaccgccgcccggcgtccaacatggacccctacgtggtcacctccatgatcgccgagaccaccatcatctggaagccctgagggctaaggcggccgttccgtcgcgttgcgttgcgttgcagggtccccgaagcgattgcaaagccattgttccttctgttccgtttgcttattattatcattattatcatctagctagatcatccgggggtcaggtcgtcgtggtgtgccaaaacagaacacagaaagaagaagaaaaaaaaacaagacgtgtggcgttaatgttatgtcatccaataatggccgtaccacacttgtggtacctttcagtaacttcgttgccggatcctctagagtcgacctgcagcccgggggatccaattcccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgtt

SEQ ID NO: 3 - DNA, 502 bp, Cassava vein mosaic virus
aaggtaattatccaagatgtagcatcaagaatccaatgtttacgggaaaaactatggaagtattatgtgagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaattt

SEQ ID NO: 4 - DNA, 462 bp, Oryza sativa
gtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtag

SEQ ID NO: 5 - DNA, 1071 bp, Zea mays
atggcctgcctcaccgacctcgtcaacctcaacctctcggacaacaccgagaagatcatcgcggaatacatatggatcggtggatctggcatggatctcaggagcaaagcaaggaccctctccggcccggtgaccgatcccagcaagctgcccaagtggaactacgacggctccagcacgggccaggcccccggcgaggacagcgaggtcatcctgtacccgcaggccatcttcaaggacccattcaggaggggcaacaacatccttgtgatgtgcgattgctacaccccagccggcgagccaatccccaccaacaagaggtacaacgccgccaagatcttcagcagccctgaggtcgccgccgaggagccgtggtatggtattgagcaggagtacaccctcctccagaaggacaccaactggccccttgggtggcccatcggtggcttccccggccctcagggtccttactactgtggaatcggcgccgaaaagtcgttcggccgcgacatcgtggacgcccactacaaggcctgcttgtatgcgggcatcaacatcagtggcatcaacggggaggtgatgccagggcagtgggagttccaagtcgggccttccgtgggtatatcttcaggcgaccaggtctgggtcgctcgctacattcttgagaggatcacggagatcgccggtgtggtggtgacgttcgacccgaagccgatcccgggcgactggaacggcgccggcgcgcacaccaactacagcacggagtcgatgaggaaggagggcgggtacgaggtgatcaaggcggccatcgagaagctgaagctgcggcacagggagcacatcgcggcatacggcgagggcaacgagcgccggctcaccggcaggcacgagaccgccgacatcaacacgttcagctggggcgtggccaaccgcggcgcgtcggtgcgcgtgggccgggagacggagcagaacggcaagggctacttcgaggaccgccgcccggcgtccaacatggacccctacgtggtcacctccatgatcgccgagaccaccatcatctggaagccctga

SEQ ID NO: 6 - DNA, 252 bp, Zea mays
gggctaaggcggccgttccgtcgcgttgcgttgcgttgcagggtccccgaagcgattgcaaagccattgttccttctgttccgtttgcttattattatcattattatcatctagctagatcatccgggggtcaggtcgtcgtggtgtgccaaaacagaacacagaaagaagaagaaaaaaaaacaagacgtgtggcgttaatgttatgtcatccaataatggccgtaccacacttgtggtacctttcagtaa

SEQ ID NO: 7 - DNA, 252 bp, Agrobacterium tumefaciens
aattcccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgtt

SEQ ID NO: 8 - DNA, 970 bp, Zea mays
tccctttaatctggcgctagatctgcatccgcggcttgcaaagataaatggcacatttagtgtgttattttgcaatacctttcatagtagatatccttaaatgcagttttaggcatgtttgggtaattaaataacatttttaggaggagttttagatttacctttctttcgtgatgactgatgacagacgtggggaattcaaatgcaactctagcgaaagttcatatatttttcataaatagctgaggctggggtaattattttttttgtagaaaaatagaataggtggaatggttggggaaggcgtaggcgctcgtggacgacgcccgataaaagacaagaggcggaattgccatgaattcgaggtagctaagtaaggcgcatatatatgccaaaaaattctactgtcactttccaatttcaatgcgctgccaaacaagccatcctggaaactgacttgaattcagcccaattctgtagatccaaacagggccggcgtcagtgcctcaggtgagagagcagcagacgatgcaaagagccaaaactgcaagcagacgcagccgaagccgaagccgaagcccaagcccaaaactgttttgtctttgcccagaaccgcgacgagcctaaactgcgcttcctcctatctacaagtccctggcacatcacgcatagtccaacctaggcgcgcaggcgataaggcgcgccacggggacgcgacatgtggtggcggacgcgatcaggatagggccaggctggccgggcgcggccacgggagaacggtggccactcgtcccacatccgcttcgtcctgtcctgtactgcgtcctgcccccaacgagagccggagccggccatcccgtcgcacactctccccctctatatatgccgtcggtgtgggggagcctactacaggacgacccaagcaagcaagcaagcagcgagtacatacatactaggcagccaggcagcc

SEQ ID NO: 9 - protein, 356 aa, Zea mays
MACLTDLVNLNLSDNTEKIIAEYIWIGGSGMDLRSKARTLSGPVTDPSKLPKWNYDGSSTGQAPGEDSEVILYPQAIFKDPFRRGNNILVMCDCYTPAGEPIPTNKRYDAAKIFSSPEVAAEEPWYGIEQEYTLLQKDTNWPLGWPIGGFPGPQGPYYCGIGAEKSFGRDIVDAHYKACLYAGINISGINGEVMPGQWEFQVGPSVGISSGDQVWVARYILERITEIAGVVVTFDPKPIPGDWNAAGAHTNYSTESMRKEGGYEVIKAAIEKLKLRHREHIAAYGEGNERRLTGRHETADINTFSWGVANRGASVRVGRETEQNGKGYFEDRRPASNMDPYVVTSMIAETTIIWKP

SEQ ID NO: 10 - protein, 356 aa, Zea mays
MACLTDLVNLNLSDTTEKIIAEYIWIGGSGMDLRSKARTLPGPVTDPSKLPKWNYDGSSTGQAPGEDSEVILYPQAIFKDPFRRGNNILVMCDCYTPAGEPIPTNKRYSAAKIFSSPEVAAEEPWYGIEQEYTLLQKDTNWPLGWPIGGFPGPQGPYYCGIGAEKSFGRDIVDAHYKACLYAGINISGINGEVMPGQWEFQVGPTVGISSGDQVWVARYILERITEIAGVVVTFDPKPIPGDWNGAGAHTNYSTESMRKEGGYEVIKAAIEKLKLRHKEHIAAYGEGNERRLTGRHETADINTFSWGVANRGASVRVGRETEQNGKGYFEDRRPASNMDPYVVTSMIAETTIIWKP

SEQ ID NO: 11 - protein, 356 aa, Zea mays
MACLTDLVNLNLSDTTEKIIAEYIWIGGSGMDLRSKARTLPGPVTDPSKLPKWNYDGSSTGQAPGEDSEVILYPQAIFKDPFRRGNNILVMCDCYTPAGEPIPTNKRYSAAKIFSSLEVAAEEPWYGIEQEYTLLQKDTNWPLGWPIGGFPGPQGPYYCGIGAEKSFGRDIVDAHYKACLYAGINISGINGEVMPGQWEFQVGPSVGISSGDQVWVARYILERITEIAGVVVTFDPKPIPGDWNGAGAHTNYSTESMRKEGGYEVIKAAIEKLKLRHKEHIAAYGEGNERRLTGRHETADINTFSWGVANRGASVRVGRETEQNGKGYFEDRRPASNMDPYVVTSMIAETTIVWKP

SEQ ID NO: 12 - protein, 356 aa, Zea mays
MACLTDLVNLNLSDTTEKIIAEYIWIGGSGMDLRSKARTLPGPVTDPSKLPKWNYDGSSTGQAPGEYSEVILYPQAIFKDPFRRGNNILVMCDCYTPAGEPIPTNKRYSAAKIFSSPELAAEEPWYGIEQEYTLLQKDTNWPLGWPIGGFPGPQGPYYCGIGAEKSFGRDIVDAHYKACLYAGINISGINGEVMPGQWEFQVGPSVGISSGDQVWVARYILERITEIAGVVVTFDPKPIPGDWNGAGAHTNYSTESMRKEGGYEVIKAAIEKLKLRHKEHIAAYGEGNERRLTGRHETADINTFSWGVANRGASVRVGRETEQNGKGYFEDRRPASNMDPYVVTSMIAETTIVWKP

SEQ ID NO: 13 - protein, 356 aa, Zea mays
MACLTDLVNLNLSDTTEKFIAEYIWIGGSGMDLRSKARTLPGPVTDPSKLPKWNYDGSSTGQAPGEDSEVILYPQAIFKDPFRRGNNILVMCDCYTPAGEPIPTNKRYSAAKIFSSPEVAAEEPWYGIEQEYTLLQKDTNWPLGWPIGGFPGPQGPYYCGIGAEKSFGRDTVDAHYKACLYAGINISGINGEVMPGQWEFQVGPSVGISSGDQVWVARYVLERITEIAGVVVTFDPKPIPGGWNGAGAHTNYSTESMRKEGGYEVIKAAIEKLKLRHKEHIAAYGEGNERRLTGRHETADINTFSWGVANRGASVRVGRETEQNGKGYFEDRRPASNMDPYVVTSMIAETTIVWKP

SEQ ID NO: 14 - protein, 355 aa, Zea mays
MACLTDLVNLNLSDTTEKIIAEYIWIGGSGMDLRSKARTLPGPVTDPSKLPKWNYDGSSTGQAPGEDSEVILYPQAIFKDPFRRGNNILVMCDCYTPAGEPIPTNKRYSAAKIFSSPEVAAEEPWYGIEQEYTLLQKDTNWPLGWPIGGFPGPQGPYYCGIGAEKSFGRDIVDAHYKACLYAGINISGINGEVMPGQWEFQVGPSVGISSGDQVWVARYILERITEIAGVVVTFDPKPIPGDWNGAGAHTNYSTESMRKEGGYEVIKAAIEKLKLRHKEHIAAYGEGNERRLTGRHETADINTFSWGVANRGASVAVGQTEQNGKGYFEDRRPASNMDPYVVTSMIAETTIVWKP

SEQ ID NO: 15 - protein, 356 aa, Setaria italica
MACLTDLVNLNLSDTTEKIIAEYIWIGGSGMDLRSKARTLSGPVTDPSKLPKWNYDGSSTGQAPGEDSEVILYPQAIFKDPFRKGNNILVMCDCYTPAGEPIPTNKRHNAAKIFSNPEVSAEEPWYGIEQEYTLLQKDTNWPLGWPVGGFPGPQGPYYCGIGADKSFGRDIVDSHYKACLYAGINISGINGEVMPGQWEFQVGPSVGISAGDQVWVARYILERITEIAGVVVTFDPKPIPGDWNGAGAHTNYSTKSMRNDGGYEVIKAAIEKLKLRHKEHIAAYGEGNERRLTGKHETADINTFSWGVANRGASVRVGRETEQNGKGYFEDRRPASNMDPYVVTSMIAETTIIWKP

SEQ ID NO: 16 - protein, 356 aa, Saccharum officinarum
MASLTDLVNLSLSDTTEKIIAEYIWIGGSGMDLRSKARTLSGPVTDPSKLPKWNYDGSSTGQAPGEDSEVILYPQAIFKDPFRRGNNILVMCDCYTPAGEPIPTNKRHNAAKIFSNPEVAAEEPWYGIEQEYTLLQKDTNWPLGWPIGGFPGPQGPYYCGIGADKSFGRDIVDAHYKACLYAGINISGINGEVMPGQWEFQVGPSVGISSGDQVWVARYILERITEIAGVVLTFDPKPIPGDWNGAGAHTNYSTKSMRNEGGYEVIKAAIEKLKLRHKEHIAAYGEGNERRLTGRHETADINTFSWGVANRGASVRVGRETEQNGKGYFEDRRPASNMDPYVVTSMIADTTILWKP

SEQ ID NO: 17 - protein, 356 aa, Oryza brachyantha
MASLTDLVNLNLSDTTEKIIAEYIWIGGSGMDLRSKARTISGPVTDPSKLPKWNYDGSSTGQAPGEDSEVILYPQAIFKDPFRRGNNILVMCDCYTPAGEPIPTNKRYNAAKIFSNPDVAAEEPWYGIEQEYTLLQKDINWPLGWPVGGFPGPQGPYYCGIGADKSFGRDIVDSHYKACLYAGINISGINGEVMPGQWEFQVGPSVGISSGDQLWVARYILERITEIAGVVVTFDPKPIPGDWNGAGAHCNYSTKSMRNDGGYEIIKSAIEKLKLRHKEHIAAYGEGNERRLTGRHETADINTFSWGVANRGASVRVGRETEQNGKGYFEDRRPASNMDPYVVTSMIAETTIIWKP

SEQ ID NO: 18 - protein, 356 aa, Oryza sativa
MASLTDLVNLNLSDTTEKIIAEYIWIGGSGMDLRSKARTLSGPVTDPSKLPKWNYDGSSTGQAPGEDSEVILYPQAIFKDPFRKGNNILVMCDCYTPAGEPIPTNKRHNAAKIFSSPEVASEEPWYGIEQEYTLLQKDINWPLGWPVGGFPGPQGPYYCGIGADKSFGRDIVDSHYKACLYAGINISGINGEVMPGQWEFQVGPSVGISAGDQVWVARYILERITEIAGVVVSFDPKPIPGDWNGAGAHTNYSTKSMRNDGGYEIIKSAIEKLKLRHKEHISAYGEGNERRLTGRHETADINTFSWGVANRGASVRVGRETEQNGKGYFEDRRPASNMDPYIVTSMIAETTIIWKP

SEQ ID NO: 19 - protein, 356 aa, Oryza glaberrima
MASLTDLVNLNLSDTTEKIIAEYIWIGGSGMDLRSKARTLSGPVTDPIKLPKWNYDGSSTGQAPGEDSEVILYPQAIFKDPFRKGNNILVMCDCYTPAGEPIPTNKRHNAAKIFSSPEVASEEPWYGIEQEYTLLQKDINWPLGWPVGGFPGPQGPYYCGIGADKSFGRDIVDSHYKACLYAGINISGINGEVMPGQWEFQVGPSVGISAGDQVWVARYILERITEIAGVVVSFDPKPIPGDWNGAGAHTNYSTKSMRNDGGYEIIKSAIEKLKLRHKEHISAYGEGNERRLTGRHETADINTFSWGVANRGASVRVGRETEQNGKGYFEDRRPASNMDPYVVTSMIAETTIIWKP

SEQ ID NO: 20 - protein, 356 aa, Brachypodium distachyon
MALLTDLLNLDLSDSTEKIIAEYIWIGGSGMDLRSKARTLSGPVTDPSKLPKWNYDGSSTGQAPGEDSEVILYPQAIFKDPFRRGNNILVMCDCYTPAGEPIPTNKRHAAAKIFSNPAVSAEEPWYGIEQEYTLLQKDINWPLGWPVGGFPGPQGPYYCSIGADKSFGRDIVDSHYKACLYAGINISGINGEVMPGQWEFQVGPSVGISAGDQVWVARYLLERITEIAGVVVSFDPKPIPGDWNGAGAHTNYSTESMRKDGGFQVIVAAVEKLKLRHKEHIAAYGEGNERRLTGKHETADIHTFSWGVANRGASVRVGRETEQNGKGYFEDRRPASNMDPYVVTAMIADTTILWKP

SEQ ID NO: 21 - protein, 356 aa, Hordeum vulgare
MALLTDLLNLDLSGSTEKIIAEYIWIGGSGMDLRSKARTLPGPVTDPSKLPKWNYDGSSTGQAPGEDSEVILYPQAIFKDPFRKGNNILVMCDCYTPAGEPIPTNKRYNAAKIFSNPDVAKEEPWYGIEQEYTLLQKDINWPLGWPVGGFPGPQGPYYCGIGADKSFGRDIVDSHYKACLFAGVNISGINGEVMPGQWEFQVGPTVGISAGDQVWVARYILERITEIAGVVVTFDPKPIPGDWNGAGAHTNYSTESMRNDGGFKVIVDAVEKLKLKHKEHIAAYGEGNERRLTGKHETADINTFSWGVANRGASVRVGRETEQNGKGYFEDRRPASNMDPYVVTSMIAETTILWKP

SEQ ID NO: 22 - protein, 356 aa, Triticum aestivum
MALLTDLLNLDLTDSTEKIIAEYIWIGGSGMDLRSKARTLPGPVTDPSKLPKWNYDGSSTGQAPGEDSEVILYPQAIFKDPFRKGNNILVMCDCYTPAGVPIPTNKRYNAAKIFSNPDVAKEEPWYGIEQEYTLLQKDINWPLGWPVGGFPGPQGPYYCSIGADKSFGRDIVDAHYKACLFAGVNISGINGEVMPGQWEFQVGPTVGISAGDQVWVARYLLERITEIAGVVVTFDPKPIPGDWNGAGAHTNYSTESMRKDGGFKVIVDAVEKLKLKHKEHIAAYGEGNERRLTGKHETADINTFSWGVANRGASVRVGRETEQNGKGYFEDRRPASNMDPYVVTSMIAETTILWKP

SEQ ID NO: 23 - protein, 356 aa, Secale cereale x Triticum turgidum subsp. durum
MALLTDLLNLDLTDSTEKIIAEYIWIGGSGMDLRSKARTLPGPVTDPSKLPKWNYDGSSTGQAPGEDSEVILYPQAIFKDPFRKGNNILVMCDCYTPAGVPIPTNKRYNAAKIFSNPDVAKEEPWYGIEQEYTLLQKDINWPLGWPVGGFPGPQGPYYCSIGADKSFGRDIVDSHYKACLFAGVNISGINGEVMPGQWEFQVGPTVGISAGDQVWVARYLLERITEIAGVVVTFDPKPIPGDWNGAGAHTNYSTESMRKDGGFKVIVDAVEKLKLKHKEHIAAYGEGNERRLTGKHETADINTFSWGVANRGASVRVGRETEQNGKGYFEDRRPASNMDPYVVTSMIAETTTLWKP

SEQ ID NO: 24 - protein, 356 aa, Lolium perenne
MALLTDLLNLDLSGSTEKIIAEYIWIGGSGMDLRSKARTLPGPVSDPSKLPKWNYDGSSTGQAPGEDSEVILYPQAIFKDPFRRGNNILVMCDCYTPAGEPIPTNKRNAAAKIFSNPAVAAEEPWYGIEQEYTLLQKDINWPLGWPVGGFPGPQGPYYCSIGAEKSFGRDIVDSHYKACLFAGINISGINGEVMPGQWEFQVGPSVGISAGDQVWVARYILERITEIAGVVVTFDPKPIPGDWNGAGAHTNYSTESMRKDGGFEVIVAAVEKLKLRHKEHIAAYGEGNERRLTGKHETADIHTFSWGVANRGASIRVGRETEQNGKGYFEDRRPASNMDPYVVTAMIADTTLLWKP

SEQ ID NO: 25 - protein, 356 aa, Secale cereale
MALLTDLLNLDLSGSTDKIIAEYIWIGGSGMDLRSKARTLPGPVTDPSKLPKWNYDGSSTGQAPGEDSEVILYPQAIFKDPFRKGNNILVMCDCYTPAGEPIPTNKRHNAAKIFSNPDVAKEEPWYGIEQEYTLLQKDINWPLGWPVGGFPGPQGPYYCGIGADKSFGRDIVDSHYKACMFAGVNISGINGEVMPGQWEFQVGPTVGISAGDQVWVARYILERITEIAGVVVTFDPKPIPGDWNGAGAHTNYSTESMRNDGGFKVIVDAVEKLKLKHKEHIAAYGEGNERRLTGKHETADINTLSWGVANRGASVRVGRETEQNGKGYFEDRRPASNMDPYVVTSMIAETTILWKP

SEQ ID NO: 26 - protein, 342 aa, Aegilops tauschii
MDVPLPYSYRQIGGSGMDLRSKARTLPGPVTDPSKLPKWNYDGSSTGQAPGEDSEVILYPQAIFKDPFRKGNNILVMCDCYTPAGVPIPTNKRYNAAKIFSNPDVAKEEPWYGIEQEYTLLQKDINWPLGWPVGGFPGPQGPYYCSIGADKSFGRDIVDSHYKACLFAGVNISGINGEVMPGQWEFQVGPTVGISAGDQVWVARYLLERITEIAGVVVTFDPKPIPGDWNGAGAHTNYSTESMRKDGGFKVIVDAVEKLKLKHKEHIAAYGEGNERRLTGKHETADINTFSWGVANRGASVRVGRETEQNGKGYFEDRRPASNMDPYVVTSMIAETTILWKP

SEQ ID NO: 27 - DNA, 1571 bp, Flaveria trinervia
aagctttactcctctcaactttcaaatcataacataaaagttcgtaggtttgtgttcttcccaaaaaaaaagtgattttttttcatcggttaattcatgattaacatttcgacattcattccactatttcacatcatgttttgatgggagattgaaatagcgataaggcgaatgtgaaagtgtgaaacaggatgagccacaccatcaccacatcacaatttacccaaataatatcccaaagattcatacgcattttgatccactgaaaccccatccaattctatccaatgcccaccacatgttcgacgatttgcctcagtgaatcaagaccaacacatgccactgctttctgctttttagtccctgataacaaacgattggctttcattgctcactgtagaaagtggagacacccaacaactatcatctccacgtggttccgtgccgcctttttgcctttcatactgctggtgcgtcatttgtcgtcatcaaagcactcacccactatcattgatctcgaaatcttgaatctttaggtttttatgctttgatacttgaactctacacacagtctcgtatctgactttttgttatctgtgttttgctttactaaagatctcacctttaatcaagttttgaacttttgatggatttgtcatgataatgaagaacacatagttattattgattatattttgacgaatcttttttcatgatcgttaaacataatttgagttctttttaccttgtctttctttgaggtttaactgtacatgaagactgtattttgagtttattgcataaatggtctatatagtttgggttaaaacaactggttttaatatcaagtttgatactagacaaaccaactttttgattaacttttaaaaaaattaataagtctatttggaaaaaaattgaaaatttgattttaaagggttaaaagttctttttgaaaagttaataagagtaacttttgaaatgtaacttttaaaaaaatactgttgataaaaaaagaaatcctaatcatgggcttagtattgtaagtagcttggatattgaagctaatttttcactttatatttatagaaaagttaatggaagtaagaggtttggatactttttttcttaatttagacgaatgttacacatgaaaaataagcgttgttttgtaagatttttttaattcgcaagcactaaactcctaatcaacttttggggttaaggagtaggcagtaaaccaaaagcgtttttgcacgatacgatgttcaaacatttgatctataacgataagtccaagtgcgttacaaaatgaaactttggtatccaatatgaaactgggtgtgtagttcagtaccaaaagcataactttcagcctccttagtgacttatgactaggcaagagaacatgtgagcccaatgtactaactttttaccccttttattaccaccaccccagccccccaccatgaaccgatcagaaaaagaagcaagaaaaacagagcattcttgctccttcttcttcatcaattcaataacattcttcataccattagaccccatcttacact

(19) United States
(12) Patent Application Publication
(10) Pub. No.: US 2018/0195083 A1
(43) Pub. Date: Jul. 12, 2018

(54) Improvement of Plant Yield

(71) Applicant: Genoplante-Valor, Paris (FR)

(72) Inventors: Bertrand Hirel, Versailles (FR); Pascual Perez, Chanonat (FR)

(73) Assignee: Genoplante-Valor, Paris (FR)

(21) Appl. No.: 15/742,378

(22) PCT Filed: Sep. 2, 2016
(86) PCT No.: PCT/EP2016/070759

§ 371 Date: Jan. 5, 2018

(30) Foreign Application Priority Data
Sep. 4, 2015 (EP) 15306362.3

(51) Int. Cl. C12N 15/82; C12N 9/00

(52) U.S. Cl. CPC C12N 15/8279; C12Y 603/01002; C12N 9/93; C12N 15/8274

(57) ABSTRACT

The invention relates to a method for improving the yield of a plant, comprising overexpressing in said plant a glutamine synthetase, said glutamine synthetase being both constitutively overexpressed in said plant and specifically overexpressed in the bundle sheath cells of said plant. The invention also relates to means for obtaining plants with improved yield.

The present invention relates to methods for controlling plant yield, in particular cereal crops, preferably maize crops, through the overexpression of glutamine synthetase (GS) activity.

The cereals including maize, wheat and rice account for 70% of worldwide food production. They require large quantities of nitrogenous fertilizers to attain maximal yields. In the past few years, there has been considerable interest in nitrogen use efficiency (NUE), which can be defined as the kernel yield per unit of nitrogen (N) in the soil and the N utilization efficiency (NutE), which is the yield per N taken up (Hirel and Lemaire, 2005a).

Using maize as a model crop, Hirel et al. (2005b) have investigated the changes in metabolite concentration and enzyme activities involved in N metabolism within a single leaf, at different stages of leaf growth and at different periods of plant development during the kernel-filling period. It was concluded that total N, chlorophyll, soluble protein content and GS activity are strongly interrelated and are indicators that mainly reflect the metabolic activity of individual leaves with regards to N assimilation and recycling, whatever the level of N fertilization.

Glutamine synthetase (GS; E.C.6.3.1.2) catalyzes the conversion of inorganic nitrogen (ammonium) into glutamine, according to the following reaction:

ATP+L-glutamate+NH₃=>ADP+phosphate+L-glutamine.

All of the nitrogen in a plant, whether derived initially from nitrate, ammonium ions, N fixation, or generated by other reactions within the plant that release ammonium (decarboxylation of glycine during photorespiration, metabolism of N transport compounds and the action of phenylalanine ammonia lyase) is channelled through the reactions catalyzed by GS.

In maize, Gln1-3 encodes the cytosolic GS isoenzyme GS1-3. The cDNA sequence of Gln1-3 is available for instance in the GenBank database under the accession number X65928; the polypeptide sequence of GS1-3 is available for instance in the UniProtKB/Swiss-Prot database under the accession number P38561. Gln1-4 encodes the cytosolic GS isoenzyme GS1-4. The cDNA sequence of Gln1-4 is available for instance in the GenBank database under the accession number X65929; the polypeptide sequence of GS1-4 is available for instance in the UniProtKB/Swiss-Prot database under the accession number P38562. The amino acid sequences of GS1-3 and GS1-4 differ in that amino acid at position 41 is a serine in GS1-3 and a proline in GS1-4, and the amino acid at position 278 is an arginine in GS1-3 and a lysine in GS1-4.

Gln1-3 gene plays a major part in the control of the number of kernels, while Gln1-4 gene plays a major part in the control of the size of the kernels. Gln1-3 and Gln1-4 act specifically on grain production, without affecting vegetative biomass production (Martin et al., 2006; International Application WO 2008/044150).

Overexpression of GS1-3 under the constitutive Cassava vein Mosaic Virus (CsVMV) promoter (Verdaguer et al., 1996) in genetically transformed maize results in a significant increase in kernel yield, in particular in kernel number, under suboptimal nitrogen feeding conditions (Martin et al., 2006; International Application WO 2008/044150). Expression of pCsVMV is constitutive and very high in mesophyll cells in planta.

He et al. (2014) have shown that overexpression of GS1-3 and GS1-4 under the constitutive actin1 promoter in genetically transformed maize results in a significant increase in yield-related traits under sufficient nitrogen conditions.

It was proposed in International Application WO 2008/044150 to produce genetically transformed maize overexpressing GS1-3 under the control of the pCsVMV promoter in the mesophyll together with GS1-4 under the control of the maize rbcS promoter (Katayama et al., 2000) in the bundle sheath cells in order to improve grain yield both in nitrogen-limiting and non-limiting conditions. However, International Application WO 2008/044150 does not describe the characterization of such a transgenic plant.

Despite the information available concerning the improvement (increase) of grain production in maize, as outlined above, there is still need for improving plant yield.

The inventors have shown in maize that the overexpression of the same glutamine synthetase of maize downstream two different promoters—a constitutive promoter and a bundle sheath cell specific promoter—leads to yield improvement (increase). More specifically, the sequence used by the inventors is SEQ ID NO: 1 (referred to as ZmGS1-b). ZmGS1-b differs from ZmGS1-3 because of three amino acids. ZmGln1-b cDNA of SEQ ID NO: 5 encodes the ZmGS1-b enzyme. The inventors have also shown a good portability of the trait “improved yield” in several genetic backgrounds.

Accordingly, the present invention provides a method for improving (increasing) the yield of a plant, wherein said method comprises overexpressing in said plant a glutamine synthetase having at least 90% identity, or by order of increasing preference at least 91%, 92%, 93%, 94%, 95%, 96%, 97%, 98%, 99% or 100% identity, with the polypeptide of SEQ ID NO: 1 (ZmGS1-b) and said glutamine synthetase being both constitutively overexpressed in said plant and specifically overexpressed in the bundle sheath cells of said plant, and provided that said glutamine synthetase constitutively overexpressed and said glutamine synthetase specifically overexpressed have the same amino acid sequence.

Unless otherwise specified, the percentage of identity between two sequences which are mentioned herein are calculated from an alignment of the two sequences over their whole length. One can use the BLAST program (Tatusova and Madden T L, 1999) with the default parameters (open gap penalty=2; extension gap penalty=5; matrix=BLOSUM 62).

The glutamine synthetase as defined above encompasses recombinant, synthetic or chimeric enzymes.

The glutamine synthetase as defined above can be from a plant of the same species as said plant in which the glutamine synthetase is overexpressed.

Advantageously, said glutamine synthetase is an autologous glutamine synthetase of said plant.

In a preferred embodiment, the glutamine synthetase is from a plant selected from the group consisting of: Zea mays, Setaria italica, Saccharum officinarum, Oryza sativa, Oryza brachyantha, Oryza glaberrima, Brachypodium distachyon, Hordeum vulgare, Triticum aestivum, Secale cereale, Secale cereale×Triticum turgidum, Lolium perenne and Aegilops tauschii, preferably Zea mays.

Advantageously the glutamine synthetase is an orthologous of ZmGS1-b (SEQ ID NO: 1) selected from the group consisting of SEQ ID NO: 9 to SEQ ID NO: 26, preferably SEQ ID NO: 9 to SEQ ID NO: 14 (glutamine synthetases from Zea mays).

As used herein the term “yield” refers to the amount of product harvested from a given acreage (e.g., weight of seeds per unit area). It is often expressed in metric quintals (1 q=100 kg) per hectare in the case of cereals. By way of example, in maize, kernel number, kernel size, kernel weight, ear length, ear diameter and ear weight are maize yield-related traits.

According to the method of the present invention the yields (e.g., grain yields when the plant is maize) are improved (increased) in said plant, both in N-limiting and non-limiting conditions.

In a preferred embodiment, the constitutively overexpressed glutamine synthetase is constitutively overexpressed in the mesophyll of said plant.

Bundle sheath cells are parenchymatous cells which surround the vascular bundle in plants. They are particularly well identified in C4 plants because of their significant role in the C4 pathway but they were also identified in C3 plants (Leegood et al., 2008). Typically, enzymes associated with sulfur assimilation are most abundant in bundle sheath cells. In addition, the p-subunit of glycine decarboxylase (GDC) is also expressed in bundle sheath cells. See for review: Berry et al., 2008; John et al., 2014.

In a preferred embodiment, the method according to the present invention applies to C4 plants.

C4 plants are well known in the art. In C4 plants, the initial fixation of atmospheric carbon dioxide occurs on phosphoenol pyruvate (a C3 compound) and is catalysed by the enzyme phosphoenolpyruvate carboxylase (PEPC). This reaction results in the formation of oxaloacetic acid (a C4 acid), hence the name C4 plants (see for review Berry et al., 2008).

Preferred C4 plants include maize (Zea mays), sorghum, sugar cane, millet and miscanthus, more preferably maize, sorghum, sugar cane, most preferably maize.

The method according to the present invention can also apply to C3 plants, such as wheat (Triticum aestivum, Triticum durum), rice and barley.

The term “overexpressing” a glutamine synthetase in a plant, herein refers to artificially increasing the quantity of said active GS enzyme produced in said plant compared to a reference plant.

A preferred method for overexpressing a glutamine synthetase comprises introducing into the genome of said plant a DNA construct comprising a nucleotide sequence encoding said glutamine synthetase, placed under the control of appropriate promoters.

The instant invention also provides means for carrying out said overexpression.

This includes in particular recombinant DNA constructs for expressing a glutamine synthetase as defined above in a host-cell, or a host organism, in particular a plant cell or a plant. These DNA constructs can be obtained and introduced in said host cell or organism by the well-known techniques of recombinant DNA and genetic engineering.

Recombinant DNA constructs of the invention include in particular an isolated polynucleotide comprising a recombinant expression cassette comprising a polynucleotide encoding a glutamine synthetase under the control of an heterologous constitutive promoter functional in a plant cell as defined above and a recombinant expression cassette comprising a polynucleotide encoding said glutamine synthetase under the control of a bundle sheath cell specific promoter, provided that both glutamine synthetase enzymes have the same amino acid sequence.

The heterologous promoter of the invention is a promoter functional in a plant cell, i.e., capable of directing transcription of a polynucleotide encoding a glutamine synthetase, as defined above, in a plant cell.

A large choice of promoters suitable for expression of genes in plants, and in particular in maize, is available in the art. They can be obtained for instance from plants, plant viruses, or bacteria such as Agrobacterium.

Constitutive promoters are promoters which are active in most tissues and cells, in particular in mesophyll cells, and under most environmental conditions. Non-limitative examples of constitutive promoters that are commonly used are the cauliflower mosaic virus (CaMV) 35S promoter, the 19S promoter (Kay et al., 1987), the Cassava vein Mosaic Virus (CsVMV) promoter (Verdaguer et al., 1996), the rice actin promoter followed by the rice actin intron (RAP-RAI; contained in the plasmid pAct1-F4) (McElroy et al., 1991), the pCRV promoter (Depigny—This et al., 1992), the ubiquitin 1 promoter of maize (Christensen et al., 1996) and the regulatory sequences of the T-DNA of Agrobacterium tumefaciens, including the mannopine synthase promoter, the nopaline synthase (Nos) promoter and the octopine synthase promoter.

Cell specific promoters are active only or mainly in certain cell types. Non-limitative examples of bundle sheath cell specific promoters that can be used in the present invention include for instance the Rubisco small subunit (rbcS) promoter (Katayama et al., 2000; SEQ ID NO: 8) or the glycine decarboxylase (GDC) p-subunit promoter (Engelmann et al., 2008; SEQ ID NO: 27).

According to a preferred embodiment, the constitutive promoter is the Cassava vein Mosaic Virus (CsVMV) promoter (e.g., SEQ ID NO: 3) and the bundle sheath cell specific promoter is the Rubisco small subunit (rbcS) promoter (e.g., SEQ ID NO: 8).

The expression cassettes generally also include a transcriptional terminator, such as the 35S transcriptional terminator or Nos terminator (Depicker et al., 1982). They may also include other regulatory sequences, such as transcription enhancer sequences.

By way of example a recombinant DNA construct according to the present invention comprises the ZmGln1-b cDNA flanked by the Cassava vein mosaic virus promoter (pCsVMV) linked to an actin intron, the ZmGln1-b terminator and the 3′Nos terminator, and another copy of the ZmGln1-b cDNA flanked by the rbcS promoter, the terminator ZmGln1-b and the 3′Nos terminator. This DNA construct is described in SEQ ID NO: 2.

Recombinant DNA constructs of the invention also include recombinant vectors containing a recombinant expression cassette comprising a polynucleotide encoding a glutamine synthetase under the control of an heterologous constitutive promoter functional in a plant cell (e.g. the CsVMV promoter) as defined above and a polynucleotide encoding said glutamine synthetase under the control of a bundle sheath cell specific promoter (e.g., the rbcS promoter), as defined above.

In a particular embodiment, the heterologous constitutive promoter functional in a plant cell is pCsVMV of SEQ ID NO: 3 and the bundle sheath cell specific promoter is proZmRbcS of SEQ ID NO: 8.

Recombinant vectors of the invention may also include other sequences of interest, such as, for instance, one or more marker genes, which allow for selection of transformed hosts.

Advantageously, the selectable marker gene is comprised between two Ds elements (i.e., transposons) in order for its removal at a later stage by interacting with the Ac transposase. This elimination system is known from one skilled in the art. By way of example, it has been described in Goldsbrough et al., 1993.

The selection of suitable vectors and the methods for inserting DNA constructs therein are well known to persons of ordinary skill in the art. The choice of the vector depends on the intended host and on the intended method of transformation of said host. A variety of techniques for genetic transformation of plant cells or plants are available in the art for many plant species. By way of non-limitative examples, one can mention virus-mediated transformation, transformation by microinjection, by electroporation, microprojectile-mediated transformation, Agrobacterium mediated transformation (Ishida et al., 1996), and the like.

The invention also comprises host cells containing a recombinant DNA construct of the invention. These host cells can be prokaryotic cells or eukaryotic cells, in particular plant cells, and preferably maize cells.

The invention also provides a method for producing a transgenic plant, in particular a maize plant, having an improved (increased) yield. Said method comprises transforming a plant cell (e.g., a maize cell) by a DNA construct of the invention and regenerating from said plant cell (e.g., maize cell) a transgenic plant (e.g., maize plant) overexpressing a glutamine synthetase under the control of a heterologous constitutive promoter functional in a plant cell as defined above and said glutamine synthetase under the control of a bundle sheath cell specific promoter, as defined above.

According to a preferred embodiment or the method of the invention, it comprises transforming a plant cell, in particular a maize cell, by a recombinant vector of the invention comprising a polynucleotide encoding a glutamine synthetase under the control of an heterologous constitutive promoter functional in a plant cell as defined above, and by a recombinant vector of the invention comprising a polynucleotide encoding said glutamine synthetase under the control of a bundle sheath cell specific promoter, as defined above, and regenerating from said plant cell (e.g., maize cell) a transgenic plant (e.g., maize plant) overexpressing both glutamine synthetase enzymes.

According to another preferred embodiment or the method of the invention, it comprises transforming a plant cell, in particular a maize cell, by a recombinant vector of the invention comprising a polynucleotide encoding a glutamine synthetase under the control of an heterologous constitutive promoter functional in a plant cell as defined above and a polynucleotide encoding said glutamine synthetase under the control of a bundle sheath cell specific promoter, as defined above, and regenerating from said plant cell (e.g., maize cell) a transgenic plant (e.g., maize plant) overexpressing both glutamine synthetase enzymes.

The invention also comprises plants genetically transformed by a recombinant DNA construct of the invention, and overexpressing a glutamine synthetase under the control of a heterologous constitutive promoter functional in a plant cell as defined above and said glutamine synthetase under the control of a bundle sheath cell specific promoter, as defined above. Preferably, said plants are transgenic maize plants, obtainable by a method of the invention, overexpressing a glutamine synthetase under the control of a heterologous constitutive promoter functional in a plant cell as defined above and said glutamine synthetase under the control of a bundle sheath cell specific promoter, as defined above. In said transgenic plants a DNA construct of the invention is comprised in a transgene integrated (i.e., stably integrated) in the plant genome, so that it is passed onto successive plant generations. Thus the transgenic plants of the invention include not only the plants resulting from the initial transgenesis, but also their descendants, as far as they contain a recombinant DNA construct of the invention. The overexpression of a glutamine synthetase under the control of a heterologous constitutive promoter functional in a plant cell as defined above and said glutamine synthetase under the control of a bundle sheath cell specific promoter, as defined above in said plants provides them with an improved (increased) yield, when compared with a plant devoid of said transgene(s).

Accordingly, the invention provides a transgenic plant or an isolated organ or tissue thereof comprising, stably integrated in its genome, a recombinant expression cassette comprising a polynucleotide encoding a glutamine synthetase under the control of a heterologous constitutive promoter functional in a plant cell as defined above and a polynucleotide encoding said glutamine synthetase under the control of a bundle sheath cell specific promoter, as defined above.

The invention also encompasses isolated organs or tissues of said transgenic plants (such as seeds, leafs, flowers, roots, stems, ears) containing a recombinant expression cassette of the invention.

Foregoing and other objects and advantages of the invention will become more apparent from the following detailed description and accompanying drawings. It is to be understood however that this foregoing detailed description is exemplary only and is not restrictive of the invention.

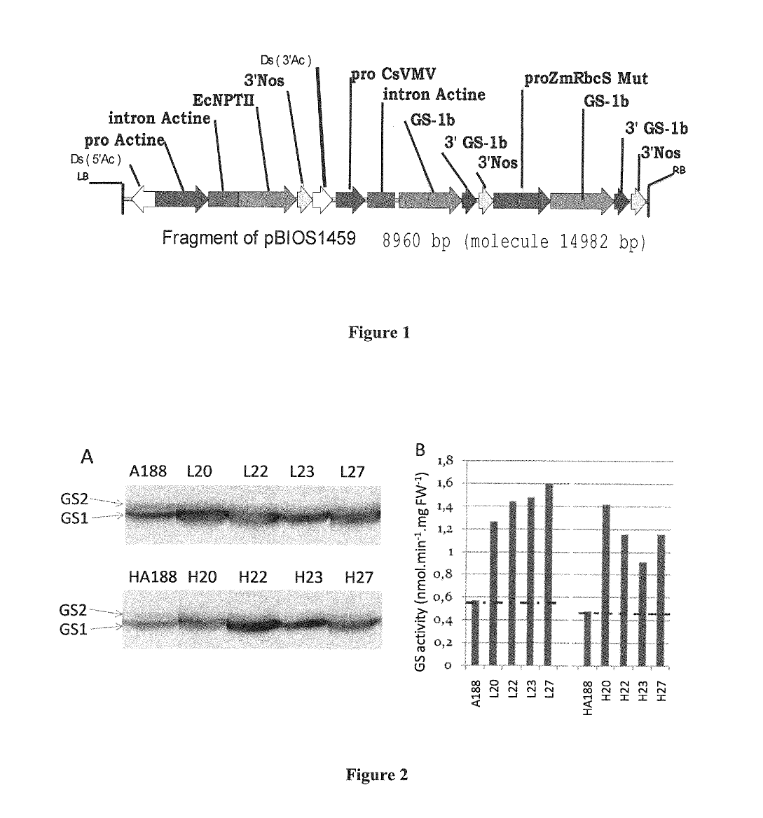

FIG. 1: Schematic representation of the T-DNA fragment introduced into maize plants. RB and LB represent the right and left borders of the T-DNA. Ds (3′Ac) and Ds (5′Ac) represent the Ds transposable elements used to further remove the selection marker conferring resistance to kanamycin. NptII is the neomycin phosphotransferase gene with the Actin promoter and the Actin intron and nopaline synthetase terminator (3′Nos) conferring kanamycin resistance. An Actin intron was placed between the maize ZmGln1-b cDNA and the CsVMV promoter.

FIG. 2: Western-blot (A) using tobacco antibodies recognizing GS1 and GS2, or enzymatic activity (B) analyses on leaf or root samples. The upper band seen on the western-blot corresponds to the plastidic GS (GS2) subunit, and the lower band corresponds to the cytosolic GS (GS1) subunit. (A) Western-blot analysis of GS1-b transformed maize lines (LXX) or from corresponding hybrid lines (HYY), leaf samples. (B) Enzymatic assays with samples of GS1-b transformed maize lines. A188: untransformed control line HA188: corresponding hybrid from untransformed A188 line.

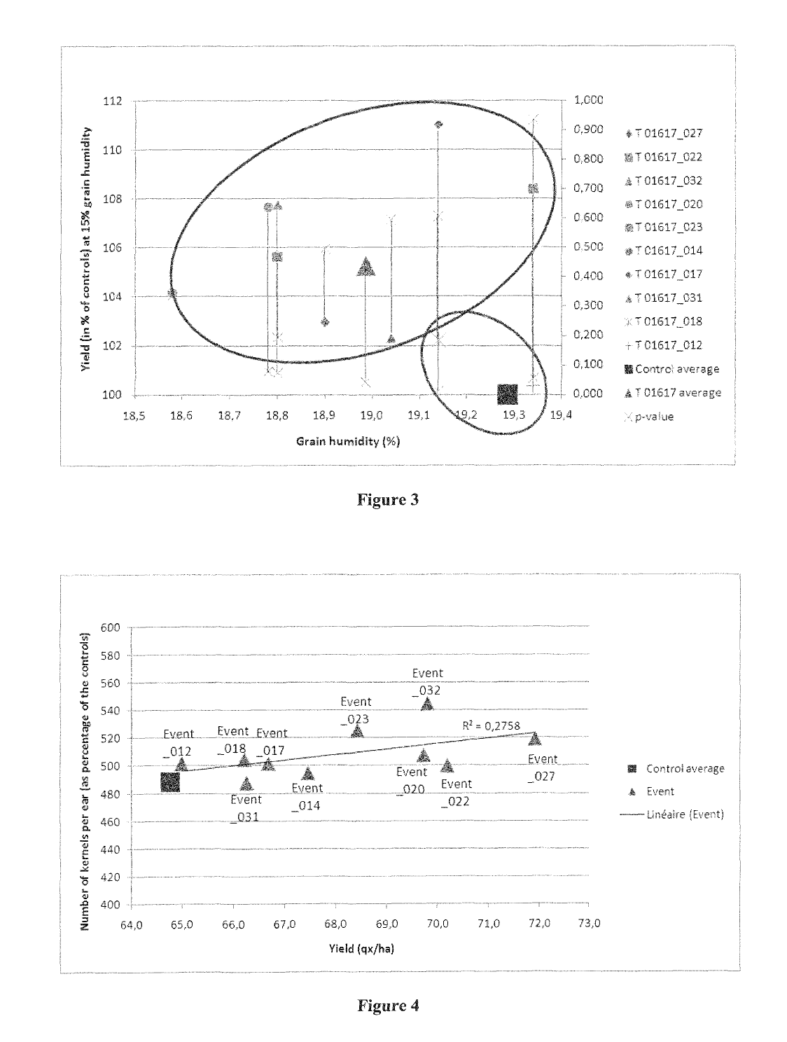

FIG. 3: Yield data and grain humidity of GS1-b over-expressing lines. The yield data is expressed as percentage of the control lines and has been normalized to grain moisture at 15%. The larger symbols correspond to the average value obtained with all transformed lines (large triangle) or with the control lines (large square). For each data point calculated, the corresponding p-value is also indicated by a cross. The p-value scale is on the left side of the figure.

FIG. 4: Kernel number per ear and yield data of GS1-b over-expressing lines. The kernel number per ear is expressed as percentage of the control lines. The yield data, normalized to grain moisture at 15%, is expressed as quintal per hectare.

### EXAMPLE 1: OVEREXPRESSION OF GS1-B (SEQ ID NO: 1) IN LEAF MESOPHYLL AND BUNDLE SHEATH CELLS IN MAIZE

The strategy, described below, is to transform maize with a T-DNA expressing a copy of the Gln1-b cDNA under control of the pCsVMV promoter, together with another copy of the Gln1-b cDNA under control of the maize bundle sheath cell specific promoter of the Rubisco (rbcS) gene (Katayama et al., 2000), in order to obtain transgenic maize plants expressing GS1-b both in the mesophyll and in the bundle sheath cells.

Plant Transformation, Regeneration and Characterization

Maize transformation of the inbred line A188 with Agrobacterium tumefaciens strain LBA4404 harbouring a super-binary plasmid was performed essentially as described by Ishida et al., 1996. In particular, the composition of all media cited hereafter is detailed in this reference. The protocol was slightly modified concerning the selective marker, which was the NPTII gene instead of the bar gene.

Super-Binary Plasmid pRec 1459

The super-binary plasmid used for transformation was the result of a recombination between plasmid pBIOS 1459 and the plasmid pSB1 (harbouring the virB and virG genes isolated from the super-virulent strain A281) within the Agrobacterium strain LBA4404 (pSB1) (Komari et al., 1996) forming the plasmid pRec 1459. pBIOS 1459 is a derivative of pSB11 (Komari et al., 1996) harbouring between the T-DNA borders, a neomycin resistance cassette (NPTII gene) (Bevan et al., 1992; Berg and Berg, 1983) flanked by an actin promoter (McElroy et al., 1990) and 3′Nos terminator (Depicker et al., 1982;), and the ZmGln1-b cDNA (Sakakibara et al., 1992, SEQ ID NO: 5) flanked by the Cassava vein mosaic virus promoter (pCsVMV) (Verdaguer et al., 1996; SEQ ID NO: 3) linked to an actin intron (McElroy et al., 1990; SEQ ID NO: 4), the ZmGln1-b terminator (SEQ ID NO: 6) and 3′Nos terminator (SEQ ID NO: 7), and another copy of the ZmGln1-b cDNA (SEQ ID NO: 5) flanked by the rbcS promoter (SEQ ID NO: 8), the ZmGln1-b terminator (SEQ ID NO: 6) and 3′Nos terminator (SEQ ID NO: 7). The resulting nucleic sequence proCsVMV-OsActin_intron-ZmGln1-b-terminator ZmGln1-b-terminator NOS-proZmRbcS-ZmGln1-b -terminator ZmGln1-b-terminator NOS is referred to as SEQ ID NO: 2. The resulting agrobacterial strain used for transformation was LB4404 (pRec 1459). pRec 1459 is schematized in FIG. 1.

Transgenic Plants

Plant transformation was conducted using immature maize embryos isolated at 10 days after pollination. Immature embryos were incubated for 5 min with A. tumefaciens and cultured for 3 days on a LSAs medium without antibiotic selection in the dark at 25° C. Upon transfer to the LSD5 medium, A. tumefaciens was counter-selected by the presence of 250 mg L-1 cefotaxime, and the transformed calli were selected by the presence of 50 mg mL-1 kanamycin. After 2 weeks of culture, developing calli were transferred to LSD10 medium containing 50 mg mL-1 kanamycin and grown for 3 weeks. Type I calli were excised and cultured for another 3 weeks on kanamycin. For regeneration, well-developed type I calli were cultured on LSZ medium at 22° C. under continuous selective pressure and on kanamycin. After 2 weeks, calli bearing shoots were transferred to RMG2 medium and cultured another 2 weeks to allow the development of roots before the transfer of the plantlets to soil and gradual acclimatization to ambient humidity. Plants were then cultivated in a glasshouse (18° C.-24° C.) and selfed or pollinated with line A188 to produce seeds.

A number of transgenic lines were selected and tested by Quantitative PCR for the number of inserted T-DNA copies and by conventional PCR for the integrity of the inserted T-DNA. Only plants with up to 2 full length T-DNA copies were kept for further analysis.

Biochemical Characterization of Transgenic Plants Over-Expressing GS1-b

The presence of the introduced GS1-b protein was determined by western-blot (FIG. 2A) using tobacco antibodies recognizing GS1 and GS2, or enzymatic activity (FIG. 2B) analyses on leaf or root samples of greenhouse grown plants.

It can be seen that leaves of GS1-b transformed lines contain a higher level of GS1 proteins, as determined by western-blot analysis, and that GS1 enzymatic activity levels are higher (more than a 2 fold increase) than those of control lines. The same results hold true for the derived hybrids.

Field Trials

Hybrids with a tester line were obtained from T3 plants issued from the selected GS1-b over-expressing transgenic maize lines.

The transformant (T0) plants were first crossed with the A188 line thereby producing T1 plants. T1 plants were then self-pollinated twice, producing T3 plants which are homozygous lines containing the transgene. These T3 plants were then crossed with the tester line thereby leading to a hybrid. This hybrid is at a T4 level with regards to the transformation step and is hemizygous for the transgene. These hybrid plants are used in field experiments.

Control Hybrids are Obtained as Follows:

Control Equiv corresponds to a cross between A188 line (the line used for transformation) and the tester line.

Control HNS corresponds to a cross between a null segregant (isolated after the second self-pollination of the T1 plants) and the tester line. Said null segregant is a homozygous line which does not bear the transgene. Although the null segregant theoretically presents the same genome as A188, it has undergone in vitro culture (via the steps of callus differentiation and regeneration) and may thus present mutations (either genetic or epigenetic) with regards to an A188 line that has not undergone in vitro culture.

Yield was calculated as follows:

During harvest, grain weight and grain moisture are measured using an on-board equipment on the combine harvester. Grain weight is then normalized to moisture at 15%, using the following formula:

Normalized grain weight=measured grain weight×(100−measured moisture (as a percentage))/85 (which is 100−normalized moisture at 15%).

As an example, if the measured grain moisture is 25%, the normalized grain weight will be: normalized grain weight=measured grain weight×75/85.

Yield is then expressed in a conventional unit (such as quintal per hectare).

Experimental Design:

The experimental block comprised 5 replicates. Each replicate comprised about 58.5 plants per plot at a density of 69600 plants/ha.

Two controls were used in this experiment as described above (a null segregant (HNS) and a control equivalent (A188 crossed with the tester line).

Grain moisture, thousand kernel weight yield and ear kernel number data are represented in the Table 1 below.

TABLE 1
Grain moisture, TKW yield and ear kernel number measured for different transformation events with
the ZmGln1-b cDNA. Data is given as per se or as a percentage compared to the control sample.

| | | | | | | | | | | | Kernel | |
|---|---|---|---|---|---|---|---|---|---|---|---|---|
| | | Grain | P-value | | | P-value | | | P-value | | number | P-value |
| | | moisture | compared | Thousand | TKW | compared | | Yield | compared | | per ear | compared |
| | Grain | as % of | to the | Kernel | as % of | to the | | as % of | to the | Number of | as % of | to the |
| | moisture | control | control | Weight | control | control | Yield | control | control | Kernels | control | control |
| Sample | (%) | average | average | (TKW) (g) | average | average | (Qx/Ha) | average | average | per ear | average | average |
| Control Equiv | 19.3 | | | 267.8 | | | 64.8 | | | 480 | | |
| T01617_027 | 19.1 | 99 | 0.7366 | 284.4 | 103 | 0.401 | 71.9 | 111 | 0.012 | 519 | 106 | 0.0679 |
| T01617_022 | 19.3 | 100 | 0.8984 | 267.6 | 97 | 0.313 | 70.2 | 108 | 0.055 | 500 | 102 | 0.4971 |
| T01617_032 | 18.8 | 97 | 0.2621 | 272.0 | 98 | 0.598 | 69.8 | 108 | 0.075 | 544 | 111 | 0.0008 |
| T01617_020 | 18.8 | 97 | 0.2431 | 279.0 | 101 | 0.806 | 69.7 | 108 | 0.079 | 507 | 104 | 0.2527 |
| T01617_023 | 18.8 | 97 | 0.2621 | 282.8 | 102 | 0.507 | 68.4 | 106 | 0.194 | 525 | 108 | 0.0294 |
| T01617_014 | 18.6 | 96 | 0.1044 | 283.4 | 102 | 0.465 | 67.5 | 104 | 0.338 | 495 | 101 | 0.7142 |
| T01617_017 | 18.9 | 98 | 0.3727 | 284.2 | 103 | 0.413 | 66.7 | 103 | 0.495 | 501 | 103 | 0.4316 |
| T01617_031 | 19.0 | 99 | 0.5700 | 270.6 | 98 | 0.496 | 66.3 | 102 | 0.596 | 487 | 100 | 0.9554 |
| T01617_018 | 19.1 | 99 | 0.7366 | 274.8 | 99 | 0.827 | 66.2 | 102 | 0.607 | 504 | 103 | 0.3380 |
| T01617_012 | 19.3 | 100 | 0.8984 | 279.6 | 101 | 0.755 | 65.0 | 100 | 0.935 | 501 | 103 | 0.4328 |
| Control HNS | 19.3 | | | 285.8 | | | 64.8 | | | 497 | | |
| Control | 19.3 | 100 | | 276.8 | | | 64.8 | 100 | | 488 | 100 | |
| average | | | | | | | | | | | | |
| T01617 | 19.0 | 98 | | 277.8 | | | 68.2 | 105 | 0.041 | 508 | 104 | 0.0417 |
| average | | | | | | | | | | | | |

FIG. 3 represents the yield expressed as percentage of the control samples and in function of grain moisture.

Improved yield (between 5 and 10% improvement compared to the controls) was observed for the hybrid plants of most GS1-b over-expressing lines. Grains from GS1-b over-expressor plants were also less humid than grains from control plants (in average 19% vs 19.3%). The transgenic lines can be grouped by those leading to a yield increase (large circle) and those given similar values to the control samples (small circle).

FIG. 4 represents the ear kernel number expressed as percentage of the control samples and in function of yield.

Improved yield may thus be explained by the increased number of kernels per ear of the transgenic plants (see FIG. 4), since a correlation between yield and ear kernel number can be seen.

### EXAMPLE 2: PORTABILITY OF THE TRAIT “IMPROVED YIELD” IN SEVERAL HYBRID BACKGROUNDS

The portability of the trait “improved yield” was tested in several hybrid backgrounds.

Hybrids were obtained from T3 plants issued from the selected GS1-b over-expressing transgenic maize lines obtained in Example 1 above crossed with either the tester line 1 (Stiff Stalk heterotic group, same tester line used in the previous example), or the tester line 2 (OH43 heterotic group) or the tester line 3 (Iodent heterotic group).

The transformed (T0) plants were first crossed with the A188 line thereby producing T1 plants. T1 plants were then self-pollinated twice, producing T3 plants which are homozygous lines containing the transgene. These T3 plants were then crossed with the tester line 1 or the tester line 2 or the tester line 3 thereby leading to a hybrid. These hybrids are at the T4 level with regards to the transformation step and are hemizygous for the transgene. These hybrid plants are used in field experiments.

Control hybrids are obtained as follows:

Control Equivalent corresponds to a cross between A188 line (the line used for transformation) and the tester line 1 or the tester line 2 or the tester line 3.

The yield was calculated as described in Example 1.

Experimental Design:

The experimental block comprised 5 replicates. Each replicate comprised about 58.5 plants per plot at a density of 69600 plants/ha.

One control was used in this experiment as described above (a control equivalent: A188 crossed with the tester line 1 or 2 or 3).

Yield measurements are represented in the Table 2 below.

TABLE 2
Yield comparison between transgenic plants and controls.

| | Mean square (Qx/ha) | | | |
|---|---|---|---|---|
| Sample | T 01617 | Control Equiv | Percentage % | Prob. > F |
| Across tester | 69.16 | 64.41 | 107.4 | 0.0106 |
| Tester 2 | 72.29 | 68.04 | 106.2 | 0.1840 |
| Tester 3 | 73.40 | 68.40 | 107.3 | 0.1136 |
| Tester 1 | 61.80 | 56.78 | 108.8 | 0.1124 |

Yield improvement (increase) of more than 7% across testers was observed showing a good portability of the trait in several genetic backgrounds.

### EXAMPLE 3: COMPARISON OF THE YIELD IMPROVEMENT BETWEEN A MAIZE TRANSGENIC PLANT OVEREXPRESSING GS1-B (SEQ ID NO: 1) IN LEAF MESOPHYLL AND BUNDLE SHEATH CELLS VERSUS A MAIZE TRANSGENIC PLANT OVEREXPRESSING GS1-B IN LEAF MESOPHYLL AND A MAIZE TRANSGENIC PLANT OVEREXPRESSING GS1-B IN BUNDLE SHEATH CELLS

Maize transgenic plants overexpressing GS1-b of SEQ ID NO: 1 in leaf mesophyll and bundle sheath cells are obtained according to the method described in Example 1.

Maize transgenic plants overexpressing GS1-b of SEQ ID NO: 1 in leaf mesophyll are obtained by transforming a maize plant with a T-DNA expressing a copy of the Gln1-b cDNA under control of the pCsVMV promoter. Maize transgenic plants overexpressing GS1-b of SEQ ID NO: 1 in bundle sheath cells are obtained by transforming a maize plant with a T-DNA expressing a copy of the Gln1-b cDNA under control of the maize bundle sheath cell specific promoter of the Rubisco (rbcS) gene (Katayama et al., 2000). The plant transformation, regeneration and biochemical characterization are carried out according to the method of Example 1. The plasmid used for the transformation of a maize plant with a T-DNA expressing a copy of the Gln1-b cDNA under control of the pCsVMV promoter comprises the ZmGln1-b cDNA (Sakakibara et al., 1992, SEQ ID NO: 5) flanked by the Cassava vein mosaic virus promoter (pCsVMV) (Verdaguer et al., 1996; SEQ ID NO: 3) linked to an actin intron (McElroy et al., 1990; SEQ ID NO: 4), the ZmGln1-b terminator (SEQ ID NO: 6) and 3′Nos terminator (SEQ ID NO: 7). The plasmid used for the transformation of a maize plant with a T-DNA expressing a copy of the Gln1-b cDNA under control of the maize bundle sheath cell specific promoter of the Rubisco (rbcS) gene comprises the ZmGln1-b cDNA (SEQ ID NO: 5) flanked by the rbcS promoter (SEQ ID NO: 8), the ZmGln1-b terminator (SEQ ID NO: 6) and 3′Nos terminator (SEQ ID NO: 7).

The field trials are carried out according to the method of Example 1. Hybrids are obtained from T3 plants issued from the selected GS1-b over-expressing transgenic maize lines crossed with a tester line, according to the method of Example 1. The control hybrids and the experimental design are also carried out according to the method of Example 1.

The yield is calculated as described in Example 1 and compared between the different transgenic hybrid maize plants.

### REFERENCES

Berg and Berg, Biotechnol., 1: 417-435, 1983.
Berry et al., Kranz Anatomy and the C4 Pathway, In: Encyclopedia of Life Sciences. John Wiley & Sons, Ltd: Chichester, 2008.
Bevan et al., Biotechnol., 24: 367-70, 1992.
Christensen et al., Transgenic. Res., 5: 213, 1996.
Depicker et al., J. Mol. Appl. Genet., 1: 561-73, 1982.
Depigny—This et al., Plant Molecular Biology, 20: 467-479, 1992.
Engelmann et al., Plant Physiol, 246: 1773-1785, 2008.
Goldsbrough et al., Nature Biotechnology, 11: 1286-1292, 1993.
He et al., Maydica electronic publication, 59: 250-256, 2014.
Hirel and Lemaire, A. Basra, and S. Goyal, eds. (Haworth's Food Product Press. Binghamton, New-York), 15: 213-257, 2005a.
Hirel et al., Physiol. Plant, 124: 178-188, 2005b.
Ishida et al., Nat. Biotechnol., 14: 745-750, 1996.
John et al., Plant Physiol, 165: 62-75, 2014
Katayama et al., Plant Mol. Biol., 44: 99-106, 2000.
Kay et al., Science, 236: 1299-1302, 1987.
Komari et al., Plant J., 10: 165-74, 1996.
Leegood et al., Journal of Experimental Botany, 59: 1663-1673, 2008.
Martin et al., Plant Cell, 18: 3252-74, 2006.
McElroy et al., Plant Cell, 2: 163, 1990.
McElroy et al., Mol. Gen. Genet., 231: 150-160, 1991.
Sakakibara et al., Plant Cell Physiol., 33: 1193-1198, 1992.
Tatusova and Madden., FEMS Microbiol. Lett., 174: 247-250, 1999.
Verdaguer et al., Plant Mol. Biol., 6: 1129-39, 1996.

1. A method of improving the yield of a plant, wherein said method comprises overexpressing in said plant a glutamine synthetase having at least 90% identity with the polypeptide of SEQ ID NO: 1 and, said glutamine synthetase being both constitutively overexpressed in said plant and specifically overexpressed in the bundle sheath cells of said plant, wherein said glutamine synthetase constitutively overexpressed and said glutamine synthetase specifically overexpressed have the same amino acid sequence.

2. The method according to claim 1, wherein said glutamine synthetase is from a plant selected from the group consisting of Zea mays, Setaria italica, Saccharum officinarum, Oryza sativa, Oryza brachyantha, Oryza glaberrima, Brachypodium distachyon, Hordeum vulgare, Triticum aestivum, Secale cereale, Secale cereale×Triticum turgidum, Lolium perenne and Aegilops tauschii.

3. The method according to claim 1, wherein said glutamine synthetase is selected from the group consisting of SEQ ID NO: 1 and SEQ ID NO: 9 to SEQ ID NO: 26.

4. The method according to claim 1, wherein said plant is a C4 plant.

5. The method according to claim 1, wherein said plant is maize and said glutamine synthetase has the amino acid sequence SEQ ID NO: 1 (ZmGS1-b).

6. An isolated polynucleotide comprising a recombinant expression cassette comprising a polynucleotide encoding a glutamine synthetase as defined in claim 1 under the control of an heterologous constitutive promoter functional in a plant cell and a recombinant expression cassette comprising a polynucleotide encoding a glutamine synthetase as defined in claim 1 under the control of a bundle sheath cell specific promoter, provided that both glutamine synthetase enzymes have the same amino acid sequence.

7. The isolated polynucleotide comprising a recombinant expression cassette according to claim 6 wherein the heterologous constitutive promoter functional in a plant cell is SEQ ID NO: 3 (pCsVMV) and the bundle sheath cell specific promoter is SEQ ID NO: 8 (proZmRbcS).

8. A recombinant vector comprising a polynucleotide of claim 6.

9. A host cell comprising:
a polynucleotide of claim 6.

10. The host cell according to claim 9 which is a plant cell.

11. The host cell according to claim 10 which is a maize cell.

12. A method of producing a transgenic plant having an improved seed yield, wherein said method comprises:
providing the plant cell of claim 10;
regenerating from said plant cell a transgenic plant overexpressing a glutamine synthetase having at least 90% identity with the polypeptide of SEQ ID NO: 1.

13. The method according to claim 12, wherein the transgenic plant is a C4 plant.

14. A transgenic plant, or an isolated organ or tissue thereof comprising, stably integrated in its genome, a recombinant expression cassette comprising a polynucleotide encoding a glutamine synthetase as defined in claim 1 under the control of an heterologous constitutive promoter functional in a plant cell and a recombinant expression cassette comprising a polynucleotide encoding a glutamine synthetase as defined in claim 1 under the control of a bundle sheath cell specific promoter, provided that both glutamine synthetase enzymes have the same amino acid sequence.

15. The transgenic plant according to claim 14, wherein said plant is a C4 plant.

16. Seed or kernel containing a recombinant expression cassette comprising a polynucleotide encoding a glutamine synthetase having at least 90% identity with the polypeptide of SEQ ID NO: 1 under the control of an heterologous constitutive promoter functional in a plant cell and a recombinant expression cassette comprising a polynucleotide encoding a glutamine synthetase having at least 90% identity with the polypeptide of SEQ ID NO: 1 under the control of a bundle sheath cell specific promoter, wherein both glutamine synthetase enzymes have the same amino acid sequence, obtained from a transgenic plant of claim 14.

17. A host cell comprising a recombinant expression cassette comprising a polynucleotide encoding a glutamine synthetase as defined in claim 1 under the control of an heterologous constitutive promoter functional in a plant cell and a recombinant expression cassette comprising a polynucleotide encoding a glutamine synthetase as defined in claim 1 under the control of a bundle sheath cell specific promoter, provided that both glutamine synthetase enzymes have the same amino acid sequence.

18. A host cell comprising the recombinant vector of claim 8.

19. The host cell according to claim 10, wherein the plant cell is a C4 plant cell.
